(19) United States
(12) Patent Application Publication
(10) Pub. No.: US 2015/0246058 A1
(43) Pub. Date: Sep. 3, 2015

(54) COMPOSITION FOR PREVENTING HAIR LOSS AND ACCELERATING HAIR GROWTH

(71) Applicant: RNS CO., LTD., Daejeon (KR)

(72) Inventor: Cheong Taek Kim, Daejeon (KR)

(73) Assignee: RNS CO., LTD., Daejeon (KR)

(21) Appl. No.: 14/379,816

(22) PCT Filed: Nov. 15, 2013
(86) PCT No.: PCT/KR2013/010405

§ 371 Date: Aug. 20, 2014

(30) Foreign Application Priority Data
Nov. 15, 2012 (KR) 10-2012-0129243

(51) Int. Cl. A61K 31/575; A61Q 7/00; A61K 31/5685; A61K 31/56; A61K 8/63

(52) U.S. Cl. CPC A61K 31/575; A61K 31/56; A61K 8/63; A61K 31/5685; A61Q 7/00

(57) ABSTRACT

Disclosed is a composition for prevention of hair loss and promotion of hair growth. The composition includes a compound represented by Formula 1, wherein A is derived from polycyclic compounds, and R is a hydroxyl group, or a saturated or unsaturated straight or branched alkyloxy or acyloxy group having 1 to 20 carbon atoms.

### TECHNICAL FIELD

The present invention relates to an agent composition for prevention of hair loss and promotion of hair growth, and more particularly, to an agent composition for preventing hair loss while promoting hair growth, which includes a nature-derived compound, as an effective ingredient, to shorten a term during which hair progresses from a telogen phase to an anagen phase among a hair growth period, thereby exhibiting remarkable effects of accelerating the growth of new hair.

### BACKGROUND ART

In general, a human has about 100,000 to 150,000 hairs. Each strand of hair has an independent period of time to live, and grows then falls out while passing through anagen, catagen and telogen phases. The above time period repeats over around 3 to 6 years and an average of 50 to 100 hairs per day typically fall out. In general, ‘hair loss’ means that a hair fraction in the anagen phase of the above time period is decreased whereas hair is increased during the catagen or telogen phases, therefore, the number of hair strands to fall out is abnormally increased.

Causes of hair loss including, for example, excess secretion of male hormone (i.e., androgen), scalp seborrheic, stress, obesity, depressed function of scalp due to peroxides, bacteria, or the like, have been investigated and/or discussed in the art. However, definite reasons for the occurrence of hair loss are still not disclosed. In recent years, there is a trend of an increase in a population troubled about or suffering from hair loss caused by a change in dietary habits, increase in stress due to social environments, etc., and the age of the population influenced by this issue has become lower. Also, a population experiencing female pattern hair loss is on the increase.

Current studies have given attention to dihydrotestosterone which is a reductive substance of a male hormone, that is, androgen, as one of major causes for hair loss. The male hormone testosterone is converted into dihydrotestosterone by an action of 5α-reductase, and it has been reported that 5α-reductase shows increased activity in hair follicles of a patient suffering from hair loss, hence causing hair loss.

In order to eliminate such causes of hair loss as described above, Merck Co. has developed a medicine that inhibits the activity of 5α-reductase to control generation of dihydrotestosterone, called ‘finasteride,’ and put the same on the market, however, several side effects of this medicine have been reported.

Accordingly, the present inventors have conducted a number of comparative experiments with a variety of compounds in order to investigate whether the compounds exhibit effects of inhibiting the activity of 5α-reductase to promote the generation of dihydrotestosterone, with the intention to develop a novel composition with effects of preventing hair loss while promoting hair growth.

### SUMMARY OF THE INVENTION

### Problems to be Solved by the Invention

It is an object of the present invention to provide a composition for preventing hair loss and promoting hair growth.

Another object of the present invention is to provide a composition capable of efficiently inhibiting 5a-reductase which induces hair loss, to thus prevent hair loss while promoting hair growth.

### Means for Solving the Problems

The above objects of the present invention will be achieved by the following means:

(1) A composition for prevention of hair loss and promotion of hair growth, including: at least one selected from compounds represented by Formula 1 below as an effective ingredient:

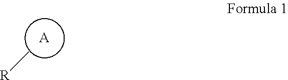

wherein

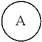

is derived from polycyclic compounds, and R is a hydroxyl group, or saturated or unsaturated straight or branched alkyloxy or acyloxy group having 1 to 20 carbon atoms.

(2) The composition according to above (1), wherein the compound represented by Formula 1 is represented by Formula 2 below,

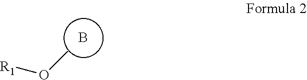

wherein

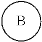

has a mother nucleus structure of at least four (4) rings linked together, and R1 is hydrogen, or a saturated or unsaturated C₁₋₃₀ straight or branched alkyl or acyl group.

(3) The composition according to above (1), wherein the compound represented by Formula 1 is at least one selected from a group consisting of) β-sitosteryl stearate, desmosteryl stearate, stigmasteryl stearate, campesteryl stearate, brassicasteryl stearate, fucosteryl stearate, cholesteryl stearate, stearoyl dehydroandrosterone, stearoyl α-amyrin, stearoyl β-amyrin, stearoyl α-boswellic acid, stearoyl β-boswellic acid, stearoyl ursolic acid, stearoyl oleanolic acid, stearoyl glycyrrhetinic acid and stearoyl betulinic acid.

(4) The composition according to above (1), wherein the compound represented by Formula 1 is contained in an amount of 0.0001 to 10 wt. % to a total weight of the composition.

### Effect of the Invention

The composition according to the present invention may efficiently inhibit 5α-reductase inducing hair loss, therefore, can be utilized as a composition for preventing hair loss and/or promoting hair growth.

### BRIEF DESCRIPTION OF THE DRAWINGS

FIG. 1 illustrates experimental results of measurement for effects of deriving a hair growth period (that is, anagen phase) and promoting hair growth when the experiment was carried out with mice [photographs five (5) weeks after application of a sample to mice]

### MODES FOR CARRYING OUT THE INVENTION

Hereinafter, the present invention will be described in more detail according to the following examples. However, these examples are proposed for illustrative purposes only and should not be construed to restrict the scope of the present invention together with appended claims thereof.

### EXAMPLE

### Examples 1 to 49

After placing compound A (a starting substance, 10 mmol) commercially available in the market in a two-neck round flask at room temperature, 50 ml of dichloromethane was introduced and the mixture was chilled using an ice bath. As a catalyst, 1 mmol of dimethyl aminopyridine and 1.1 mmol of triethylamine were further introduced, then, compound B (acyl halide, an inducing substance, 1.2 mmol) was slowly added dropwise thereto. After completion of the dropping, the mixture was allowed to undergo a reaction at room temperature. 100 ml of diluted hydrochloric acid solution was introduced into the reaction product and extraction was conducted using 200 ml of dichloromethane. After drying a dichloromethane layer under reduced pressure, separation and purification were conducted by means of silica-gel column chromatography (ethyl acetate:hexane=1:50), thereby obtaining a final product. The obtained product was identified by means of fast atom bombardment mass spectrometry (hereinafter, ‘FAB-MS’) and results thereof are shown in Table 1 below.

TABLE 1
Results of Examples 1 to 49

| | | | | Molec- |
|---|---|---|---|---|
| | Compound A | Compound B | | ular |
| Exam- | (Starting | (Inducing | Reaction | weight |
| ple | substance) | substance) | product | [M + H]⁺ |
| 1 | β-amyrin | — | β-amyrin | 427 |
| 2 | β-amyrin | acetyl | β-amyrin | 459 |
| | | chloride | acetate | |
| 3 | β-amyrin | 3-methyl-2- | β-amyrin 3- | 509 |
| | | butenoyl | methyl-2- | |
| | | chloride | butenate | |
| 4 | β-amyrin | 2-ethyl- | β-amyrin 2- | 553 |
| | | hexanoyl | ethylhexanoate | |
| | | chloride | | |
| 5 | β-amyrin | octanoyl | β-amyrin | 553 |
| | | chloride | octanoate | |
| 6 | β-amyrin | lauroyl | β-amyrin | 609 |
| | | chloride | laurate | |
| 7 | β-amyrin | palmitoyl | β-amyrin | 665 |
| | | chloride | palmitate | |
| 8 | β-amyrin | stearoyl | β-amyrin | 693 |
| | | chloride | stearate | |
| 9 | β-amyrin | behenoyl | β-amyrin | 749 |
| | | chloride | behenate | |
| 10 | α-amyrin | — | α-amyrin | 427 |
| 11 | α-amyrin | acetyl | α-amyrin | 459 |
| | | chloride | acetate | |
| 12 | α-amyrin | octanoyl | α-amyrin | 553 |
| | | chloride | octanoate | |
| 13 | α-amyrin | stearoyl | α-amyrin | 693 |
| | | chloride | stearate | |
| 14 | β-sitosterol | — | β-sitosterol | 415 |
| 15 | β-sitosterol | acetyl | β-sitosterol | 447 |
| | | chloride | acetate | |
| 16 | β-sitosterol | octanoyl | β-sitosterol | 541 |
| | | chloride | octanoate | |
| 17 | β-sitosterol | stearoyl | β-sitosterol | 681 |
| | | chloride | stearate | |
| 18 | ursolic acid | — | ursolic acid | 457 |
| 19 | ursolic acid | acetyl | ursolic acid | 489 |
| | | chloride | acetate | |
| 20 | ursolic acid | octanoyl | ursolic acid | 583 |
| | | chloride | octanoate | |
| 21 | ursolic acid | stearoyl | ursolic acid | 723 |
| | | chloride | stearate | |
| 22 | oleanolic acid | — | oleanolic acid | 457 |
| 23 | oleanolic acid | acetyl | oleanolic acid | 489 |
| | | chloride | acetate | |
| 24 | oleanolic acid | octanoyl | oleanolic acid | 583 |
| | | chloride | octanoate | |
| 25 | oleanolic acid | stearoyl | oleanolic acid | 723 |
| | | chloride | stearate | |
| 26 | α-boswellic | — | α-boswellic | 457 |
| | acid | | acid | |
| 27 | α-boswellic | acetyl | α-boswellic | 489 |
| | acid | chloride | acid acetate | |
| 28 | α-boswellic | stearoyl | α-boswellic | 723 |
| | acid | chloride | acid stearate | |
| 29 | β-boswellic | — | β-boswellic | 457 |
| | acid | | acid | |
| 30 | β-boswellic | acetyl | β-boswellic | 489 |
| | acid | chloride | acid acetate | |
| 31 | β-boswellic | stearoyl | β-boswellic | 723 |
| | acid | chloride | acid stearate | |
| 32 | betulinic acid | — | betulinic acid | 457 |
| 33 | betulinic acid | stearoyl | betulinic acid | 723 |
| | | chloride | stearate | |
| 34 | glycyrrhetinic | — | glycyrrhetinic | 471 |
| | acid | | acid | |
| 35 | glycyrrhetinic | stearoyl | glycyrrhetinic | 737 |
| | acid | chloride | acid stearate | |
| 36 | Dihydroandro- | — | dihydroandro- | 289 |
| | sterone | | sterone | |
| 37 | Dihydroandro- | stearoyl | dihydroandro- | 555 |
| | sterone | chloride | sterone stearate | |
| 38 | Desmosterol | — | desmosterol | 385 |
| 39 | Desmosterol | stearoyl | desmosterol | 651 |
| | | chloride | stearate | |
| 40 | Cholesterol | — | cholesterol | 387 |
| 41 | Cholesterol | stearoyl | cholesterol | 653 |
| | | chloride | stearate | |
| 42 | Stigmasterol | — | Stigmasterol | 413 |
| 43 | Stigmasterol | stearoyl | Stigmasterol | 679 |
| | | chloride | stearate | |
| 44 | Campesterol | — | Campesterol | 401 |
| 45 | Campesterol | stearoyl | Campesterol | 667 |
| | | chloride | stearate | |
| 46 | Brassicasterol | — | brassicasterol | 399 |
| 47 | Brassicasterol | stearoyl | brassicasterol | 665 |
| | | chloride | stearate | |
| 48 | Fucosterol | — | fucosterol | 413 |
| 49 | Fucosterol | stearoyl | fucosterol | 679 |
| | | chloride | stearate | |

### Examples 50 to 72

1.60 g of sodium hydride (60%, 40 mmol) and 200 ml of tetrahydrofuran anhydride were introduced into a 500 ml three-neck flask equipped with a thermostat and a condenser, and the mixture was agitated at room temperature. Then, compound C (a starting substance, 20 mmol) in solid state was introduced into the mixture at room temperature, followed by agitating for 30 minutes. In addition, compound D was introduced (an inducing substance, 22 mmol) into the mixture at room temperature, and agitated for 36 hours or more at a reflux temperature of tetrahydrofuran. Thereafter, 100 ml of water was added to the reaction product and extraction was carried out using 200 ml of ether. The extracted product was dried using anhydrous magnesium sulfate, and then, filtered and concentrated. Following this, separation and purification were conducted by means of silica-gel column chromatography (ethyl acetate:hexane=1:60), thereby obtaining a final product. The obtained product was identified by means of FAB-MS.

TABLE 2
Results of Examples 50 to 72

| | | | | Molec- |
|---|---|---|---|---|
| | Compound A | Compound B | | ular |
| Exam- | (Starting | (Inducing | | weight |
| ple | substance) | substance) | Reaction product | [M + H]⁺ |
| 50 | β-amyrin | methyl | O-methyl-β-amyrin | 441 |
| | | chloride | | |
| 51 | β-amyrin | pentyl | O-pentyl-β-amyrin | 497 |
| | | chloride | | |
| 52 | β-amyrin | octyl | O-octyl-β-amyrin | 539 |
| | | chloride | | |
| 53 | β-amyrin | dodecyl | O-dodecyl-β-amyrin | 595 |
| | | chloride | | |
| 54 | β-amyrin | octadecyl | O-octadecyl-β- | 679 |
| | | chloride | amyrin | |
| 55 | α-amyrin | octyl | O-octyl-α-amyrin | 539 |
| | | chloride | | |
| 56 | α-amyrin | octadecyl | O-octadecyl-α- | 679 |
| | | chloride | amyrin | |
| 57 | β-sitosterol | octyl | O-octyl-β- | 527 |
| | | chloride | sitosterol | |
| 58 | β-sitosterol | octadecyl | O-octadecyl-β- | 667 |
| | | chloride | sitosterol | |
| 59 | ursolic acid | octyl | O-octyl-ursolic | 569 |
| | | chloride | acid | |
| 60 | ursolic acid | octadecyl | O-octadecyl-ursolic | 709 |
| | | chloride | acid | |
| 61 | oleanolic acid | octyl | O-octyl-oleanolic | 569 |
| | | chloride | acid | |
| 62 | oleanolic acid | octadecyl | O-octadecyl- | 709 |
| | | chloride | oleanolic acid | |
| 63 | α-boswellic | octyl | O-octyl-α-boswellic | 569 |
| | acid | chloride | acid | |
| 64 | β-boswellic | octyl | O-octyl-β-boswellic | 709 |
| | acid | chloride | acid | |
| 65 | betulinic acid | octyl | O-octyl-betulinic | 569 |
| | | chloride | acid | |
| 66 | glycyrrhetinic | octyl | O-octyl- | 709 |
| | acid | chloride | glycyrrhetinic acid | |
| 67 | Desmosterol | octyl | O-octyl-desmosterol | 497 |
| | | chloride | | |
| 68 | Cholesterol | octyl | O-octyl-cholesterol | 499 |
| | | chloride | | |
| 69 | Stigmasterol | octyl | O-octyl- | 525 |
| | | chloride | stigmasterol | |
| 70 | Campesterol | octyl | O-octyl-campesterol | 513 |
| | | chloride | | |
| 71 | Brassicasterol | octyl | O-octyl- | 511 |
| | | chloride | brassicasterol | |
| 72 | Fucosterol | octyl | O-octyl-fucosterol | 525 |
| | | chloride | | |

### EXPERIMENTAL EXAMPLE

### Experimental Example 1

### Assessment of 5α-Reductase Inhibitory Efficacy

After seeding 1 ml of dermal papilla cells (DPCs) by 5×10⁵/ml into each well of a 24-well plate, then incubating the same for 24 hours, each of the reaction products prepared according to procedures described in Examples 1 to 72 was added in amounts of 50 μg/ml, 10 μg/ml and 0 μg/ml, respectively, to individual wells. After 1 hour, the wells were subjected to stimulation using 50 nM testosterone (DMSO dissolved). The supernatant was discarded 24 hours after culturing, to thus obtain DPc pellets. A content of dihydrotestosterone (DHT) (DB52021, Hamburg, Germany) was determined by means of enzyme-linked immune-sorbent assay. DPCs cell pellets were seeded in an amount of 100 μL into each of Dihydrotestosterone (DHT) ELISA microtiter strips coated with rabbit anti-DHT antibody, 25 μL of DHT antiserum was seeded in every well, and subjected to a reaction at 2 to 8° C. for 15 to 20 hours, followed by washing the same three times. Thereafter, 100 μL of DHT-HRP enzyme conjugate was seeded in every well and reacted on a shaker at room temperature for 30 minutes, followed by washing the same three times. Furthermore, 100 μL of substrate was seeded in every well and reacted on a shaker at room temperature for 20 to 30 minutes. Then, the product was treated using 100 μL stop solution, and subjected to measurement of absorbance at 450 nm to express an amount of produced DHT by the absorbance (three times repeated). An amount of produced DHT relative to a control was calculated in terms of an inhibitory rate (%), and results thereof are shown in Table 3 below.

TABLE 3
5α-reductase inhibitory rate of
compounds prepared in Examples 1 to 72

| | Sample | |
|---|---|---|
| Sample | concentration(μg/ml) | Inhibitory rate (%) |
| Control | — | — |
| Compound in Example 1 | 10 | 15 |
| | 50 | 28 |
| Compound in Example 2 | 10 | 21 |
| | 50 | 34 |
| Compound in Example 3 | 10 | 28 |
| | 50 | 41 |
| Compound in Example 4 | 10 | 36 |
| | 50 | 54 |
| Compound in Example 5 | 10 | 41 |
| | 50 | 57 |
| Compound in Example 6 | 10 | 43 |
| | 50 | 59 |
| Compound in Example 7 | 10 | 53 |
| | 50 | 67 |
| Compound in Example 8 | 10 | 48 |
| | 50 | 63 |
| Compound in Example 9 | 10 | 38 |
| | 50 | 59 |
| Compound in Example 10 | 10 | 13 |
| | 50 | 29 |
| Compound in Example 11 | 10 | 31 |
| | 50 | 42 |
| Compound in Example 12 | 10 | 35 |
| | 50 | 49 |
| Compound in Example 13 | 10 | 45 |
| | 50 | 57 |
| Compound in Example 14 | 10 | 11 |
| | 50 | 26 |
| Compound in Example 15 | 10 | 21 |
| | 50 | 29 |
| Compound in Example 16 | 10 | 28 |
| | 50 | 39 |
| Compound in Example 17 | 10 | 31 |
| | 50 | 42 |
| Compound in Example 18 | 10 | 16 |
| | 50 | 29 |
| Compound in Example 19 | 10 | 22 |
| | 50 | 31 |
| Compound in Example 20 | 10 | 31 |
| | 50 | 46 |
| Compound in Example 211 | 10 | 35 |
| | 50 | 51 |
| Compound in Example 22 | 10 | 7 |
| | 50 | 16 |
| Compound in Example 23 | 10 | 11 |
| | 50 | 23 |
| Compound in Example 24 | 10 | 21 |
| | 50 | 39 |
| Compound in Example 25 | 10 | 35 |
| | 50 | 41 |
| Compound in Example 26 | 10 | 10 |
| | 50 | 19 |
| Compound in Example 27 | 10 | 21 |
| | 50 | 29 |
| Compound in Example 28 | 10 | 37 |
| | 50 | 51 |
| Compound in Example 29 | 10 | 11 |
| | 50 | 23 |
| Compound in Example 30 | 10 | 14 |
| | 50 | 29 |
| Compound in Example 31 | 10 | 31 |
| | 50 | 47 |
| Compound in Example 32 | 10 | 9 |
| | 50 | 17 |
| Compound in Example 33 | 10 | 31 |
| | 50 | 51 |
| Compound in Example 34 | 10 | 6 |
| | 50 | 18 |
| Compound in Example 35 | 10 | 24 |
| | 50 | 39 |
| Compound in Example 36 | 10 | 9 |
| | 50 | 28 |
| Compound in Example 37 | 10 | 24 |
| | 50 | 38 |
| Compound in Example 38 | 10 | 12 |
| | 50 | 31 |
| Compound in Example 39 | 10 | 26 |
| | 50 | 41 |
| Compound in Example 40 | 10 | 15 |
| | 50 | 17 |
| Compound in Example 41 | 10 | 26 |
| | 50 | 39 |
| Compound in Example 42 | 10 | 8 |
| | 50 | 24 |
| Compound in Example 43 | 10 | 31 |
| | 50 | 41 |
| Compound in Example 44 | 10 | 11 |
| | 50 | 21 |
| Compound in Example 45 | 10 | 26 |
| | 50 | 43 |
| Compound in Example 46 | 10 | 8 |
| | 50 | 19 |
| Compound in Example 47 | 10 | 24 |
| | 50 | 39 |
| Compound in Example 48 | 10 | 12 |
| | 50 | 19 |
| Compound in Example 49 | 10 | 25 |
| | 50 | 41 |
| Compound in Example 50 | 10 | 16 |
| | 50 | 31 |
| Compound in Example 51 | 10 | 22 |
| | 50 | 38 |
| Compound in Example 52 | 10 | 28 |
| | 50 | 43 |
| Compound in Example 53 | 10 | 31 |
| | 50 | 41 |
| Compound in Example 54 | 10 | 35 |
| | 50 | 48 |
| Compound in Example 55 | 10 | 24 |
| | 50 | 38 |
| Compound in Example 56 | 10 | 38 |
| | 50 | 49 |
| Compound in Example 57 | 10 | 21 |
| | 50 | 41 |
| Compound in Example 58 | 10 | 31 |
| | 50 | 46 |
| Compound in Example 59 | 10 | 22 |
| | 50 | 41 |
| Compound in Example 60 | 10 | 35 |
| | 50 | 49 |
| Compound in Example 61 | 10 | 22 |
| | 50 | 37 |
| Compound in Example 62 | 10 | 29 |
| | 50 | 51 |
| Compound in Example 63 | 10 | 21 |
| | 50 | 34 |
| Compound in Example 64 | 10 | 35 |
| | 50 | 51 |
| Compound in Example 65 | 10 | 19 |
| | 50 | 31 |
| Compound in Example 66 | 10 | 33 |
| | 50 | 51 |
| Compound in Example 67 | 10 | 24 |
| | 50 | 41 |
| Compound in Example 68 | 10 | 35 |
| | 50 | 45 |
| Compound in Example 69 | 10 | 19 |
| | 50 | 33 |
| Compound in Example 70 | 10 | 28 |
| | 50 | 51 |
| Compound in Example 71 | 10 | 29 |
| | 50 | 38 |
| Compound in Example 72 | 10 | 19 |
| | 50 | 35 |

From the results shown in Table 3 above, it can be seen that the compounds represented by Formula 1 have an outstanding 5a-reductase inhibitory rate.

### Experimental Example 2

### Experimentation for Assessment of Anagen Phase Induction and Promotion

Experimentation for effects of inducing and promoting an anagen phase (that is, hair growth period) was performed with 7 weeks-old mice (C57BL/6, female) widely used in assessment of activity for anagen phase induction. First, using an electric shaver, hair on the dorsal part of a mouse was removed. Each mouse was weighed and all mice were divided into groups, each group having five (5) mice, in order to uniformly disperse body weights of the mice. Hair cut regions were coated with an excipient, control and test medicine, respectively. In this regard, the excipient was 95% ethanol, the control was 2% minoxidil, and the test medicine used herein was the compound in Example 8 diluted with 95% ethanol to have a final concentration of 0.5%. Using a brush, each of the above test samples was sufficiently applied to the dorsal part of each mouse once per day.

Overall treatment effects were determined and assessed at four levels as to whether hair growth and restoration are remarkably improved (++), improved (+), slightly improved (±) or not changed (−), respectively, 20 days after experimentation, as compared to before dosing the test sample.

TABLE 4
Results of assessment for anagen phase induction and promotion

Number of mouse

| Sample | ++ | + | ± | − |
|---|---|---|---|---|
| Excipient | — | 1 | 1 | 3 |
| Control (Minoxidil 2%) | 2 | 1 | 1 | 1 |
| Compound in Example | 3 | 1 | 1 | — |
| 8(0.5%) | | | | |

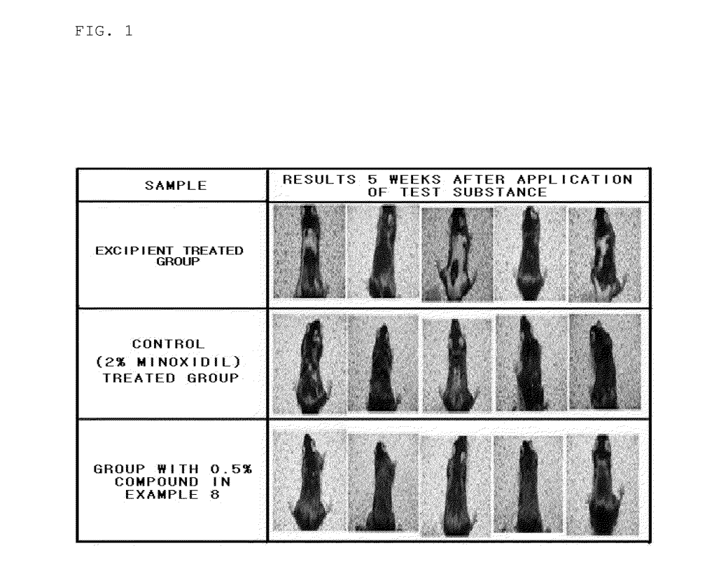

As listed in Table 4 above, it can be seen that mice with the compound in Example 8 applied thereto show rapid hair growth in the hair cut region and this result can also be seen in FIG. 1.

### BEST MODE FOR CARRYING OUT PREFERRED EMBODIMENTS OF THE INVENTION

A composition for prevention of hair loss and promotion of hair growth according to the present invention may contain at least one selected from compounds represented by Formula 1 below, as an effective ingredient:

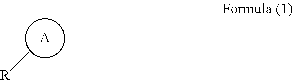

Hereinafter, the present invention will be described in more detail.

The composition for prevention of hair loss and promotion of hair growth according to an embodiment of the present invention may offer a cyclic derivative compound having a structure of Formula 1 or a pharmacologically acceptable salt thereof.

Herein,

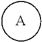

is derived from polycyclic compounds and, in particular, may be a compound composed of at least four rings.

More particularly, the above compound may include, for example, β-sitosterol, desmosterol, stigmasterol, campesterol, brassicasterol, fucosterol, cholesterol, dehydroandrosterone, α-amyrin, β-amyrin, α-boswellic acid, β-boswellic acid, ursolic acid, oleanolic acid, glycyrrhetinic acid, betulinic acid, or the like, however, not be particularly limited thereto.

R is a hydroxyl group, or a saturated or unsaturated straight or branched alkyl or acyl group having 1 to 30 carbon atoms.

The cyclic compound described above may be a saturated or unsaturated cyclic compound with a structure of four (4) or five (5) rings linked together.

In Formula 1, R is a hydroxyl group, or a saturated or unsaturated C₁₋₃₀ straight or branched alkyloxy or acyloxy group.

In Formula 1,

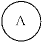

may have a mother nucleus structure formed of at least four (4) rings linked together, preferably, is a derivative of the polycyclic compound including 6-member ring or 5-member ring and contains at least one hydroxyl group. More preferably, Formula 1 may have a structure represented by Formula 2 below.

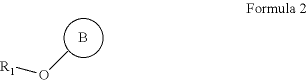

In Formula 2 above,

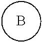

may have a mother nucleus structure of at least four (4) rings linked together, preferably, is a derivative of the polycyclic compound having a 6-member ring or 5-member ring.

In Formula 2, R1 may be hydrogen, or a saturated or unsaturated C₁₋₃₀ straight or branched alkyl or acyl group.

Hereinafter, the present invention will be described in more detail.

The compound of the present invention may be an aliphatic cyclic compound, preferably, a homocyclic compound in which at least three 5-member rings or 6-member rings are linked together, and may be prepared by dissolving a compound having a structure, wherein at least one compound is bound, in a proper organic solvent; gently dropping acyl halide or alkyl halide in a desired equivalence ratio into the above solution to allow esterification or etherification with a hydroxyl group in the cyclic structure, resulting in the above compound; or, otherwise, conducting hydrogenation of the obtained reaction product to thus offer the above compound, as shown in the following reaction schemes. In this case, the organic solvent used herein is not particularly limited but may include, for example, dichloromethane, chloroform, tetrahydrofuran, acetonitrile, dimethyl formamide, pyridine, or the like. Specifically, for the etherification, sodium hydride (NaH) or potassium carbonate (K₂CO₃) may be used as a reaction catalyst.

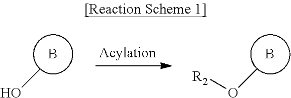

In Reaction Scheme 1 above, R₂ may be a saturated or unsaturated C₁₋₃₀ acyl group, which can be a straight or branched type.

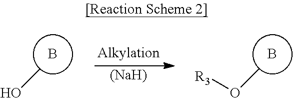

In Reaction Scheme 2 above, R₃ may be a saturated or unsaturated C₁₋₃₀ alkyl group, which can be a straight or branched type.

More particularly, the compound represented by Formula I according to the present invention may include some products prepared in case where a compound having a cyclic structure, that is,

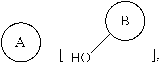

among substances formed according to Reaction Scheme 1 and Reaction Scheme 2, is derived from β-amyrin, and particular examples of the above products may be as follows.

For example, β-amyrin, O-methyl-β-amyrin, O-pentyl-β-amyrin, O-octyl-β-amyrin, O-dodecyl-β-amyrin, O-octadecyl-β-amyrin, O-docosanyl-β-amyrin, acetyl-β-amyrin, (3-methyl-2-butenoyl)-β-amyrin, 2-ethylhexanoyl-β-amyrin, octanoyl-β-amyrin, lauroyl-β-amyrin, palmitoyl-β-amyrin, stearoyl-β-amyrin, behenoyl-β-amyrin, or the like, may be included, however, the above product is not particularly limited thereto.

Further, among substances formed according to Reaction Scheme 1, particular ones obtained when R₂ is stearoyl may be exemplified below.

For example, β-sitosteryl stearate, desmosteryl stearate, stigmasteryl stearate, campesteryl stearate, brassicasteryl stearate, fucosteryl stearate, cholesterolyl stearate, stearoyl dehydroandrosterone, stearoyl α-amyrin, stearoyl β-amyrin, stearoyl α-boswellic acid, stearoyl β-boswellic acid, stearoyl ursolic acid, stearoyl oleanolic acid, stearoyl glycyrrhetinic acid, stearoyl betulinic acid, or the like, may be included, however, the above substance is not particularly limited thereto.

Further, the present invention provides a pharmaceutical composition for prevention of hair loss and promotion of hair growth, including the compound represented by Formula 1 or any pharmacologically acceptable salt thereof as an effective ingredient.

The compound represented by Formula 1 according to the present invention may be prepared by any conventional method known in the related art, in any form of pharmaceutically acceptable salts and/or solvates.

The above salt useable herein may include acid-addition salts formed using pharmaceutically acceptable free acids. Such acid-addition salts may be prepared by any conventional method, for example, including: dissolving a compound in an excess acid-containing solution to form a salt; and precipitating the salt using a water-miscible organic solvent, For example, methanol, ethanol, acetone or acetonitrile. After heating the compound and acid or alcohol (i.e., glycol monomethylether) in water in equal molar amounts, this mixture is evaporated and dried. Otherwise, precipitated salts may be treated by suction filtration.

In this case, the free acid used herein may include organic acids and inorganic acids. The inorganic acids may include, for example, hydrochloric acid, phosphoric acid, sulfuric acid, nitric acid, tartaric acid, or the like. Further, the organic acids used herein may include, for example, methane sulfonic acid, p-toluene sulfonic acid, acetic acid, trifluoroacetic acid, citric acid, maleic acid, succinic acid, oxalic acid, benzoic acid, tartaric acid, fumaric acid, mandelic acid, propionic acid, citric acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, or the like.

Further, a pharmaceutically acceptable metal salt may be prepared using a base. For example, an alkali-metal salt or alkali-earth metal salt may be prepared by dissolving a compound in an excess alkali-metal hydroxide solution or alkali-earth metal hydroxide solution, filtering an insoluble compound salt, and evaporating and drying the filtrate. In this regard, the metal salt prepared therein may be appropriately sodium, potassium or calcium-based salt in view of pharmaceutical applications. Further, silver salts corresponding to the above metal salt may be formed by reacting the alkali-metal or alkali-earth metal salt with any proper silver salt (i.e., silver nitrate).

The pharmaceutically acceptable salt represented by Formula 1 may include salts of acidic or basic groups possibly present in the compound represented by Formula 1, unless otherwise stated. More particularly, such pharmaceutically acceptable salts as described above may include, for example, sodium, calcium and potassium salts of a hydroxyl group. Alternatively, pharmaceutically acceptable salts of an amino group may include, for example, hydrobromide, sulfate, hydrosulfate, phosphate, hydrophosphate, dihydrophosphate, acetate, succinate, citrate, tartrate, lactate, mandelate, methane sulfonate (mesylate) and p-toluene sulfonate(tosylate) salts, which may be prepared according to conventional methods or processes for preparation of salts generally known in the related art.

The composition including the compound of the present invention may further include appropriately any of carriers, excipients or diluents used in conventional methods.

The carriers, excipients and diluents possibly included in the composition of the present invention may include, for example, lactose, dextrose, sucrose, sorbitol, mannitol, xylitol, erythritol, maltitol, starch, acacia rubber, alginate, gelatin, calcium phosphate, calcium silicate, cellulose, methyl cellulose, amorphous cellulose, polyvinyl pyrrolidone, water, methyl hydroxybenzoate, propyl hydroxybenzoate, talc, magnesium stearate and mineral oil.

The composition including the compound of the present invention may be formulated in a variety of forms according to any conventional method, for example: oral formulations such as powder, granules, tablets, capsules, suspension, emulsion, syrup, aerosol, etc.; topical preparations; suppositories; or sterile injectable solution, or the like.

More particularly, the formulation may be prepared using any of fillers, bulk additives, binders, humectants, disintegrants, diluents such as surfactants, or excipients generally used in the related art. In case of a solid formulation used for oral administration, tablets, pills, powder, granules, capsules, etc. may be included, and the solid formulation may be prepared by adding at least one excipient, for example, starch, calcium carbonate, sucrose, lactose, gelatin, etc. to the obtained extract. In addition to a simple excipient, a lubricant such as magnesium stearate, talc, etc. may also be used. Alternatively, a liquid formulation used for oral administration may include, for example, suspension, internal solution, emulsion, syrup, etc., and simple diluents generally used in the related art such as water, liquid paraffin, or other different excipients, for example, a wetting agent, a sweetening agent, aromatics, preservatives, or the like, may also be included. Formulations for parenteral administration may include, for example, sterile water solution, non-aqueous solvent, suspension, emulsion, lyophilized formulations and suppositories. The non-aqueous solvent or suspension used herein may include, for example, propylene glycol, polyethylene glycol, vegetable oil such as olive oil, injectable ester such as ethyloleate, or the like. Basic materials for suppositories may include, for example, witepsol, macrogol, tween 61, cacao butter, laurinum, glycero-gelatin, or the like.

The composition for prevention of hair loss and promotion of hair growth including the compound represented by Formula 1 according to the preset invention or a pharmacologically acceptable salt thereof, as an effective ingredient, may be prepared and used as a pharmaceutical composition in any type of dermal topical formulations applicable to a skin, which include, for example, cream, gel, patch, spray, ointment, solid ointment, lotion, liniment, pasta or cataplasma, or the like, however, not be particularly limited thereto.

A preferred dose of administering the compound represented by Formula 1 according to the present invention or a pharmacologically acceptable salt thereof may depend upon condition and body weight of a patient, severity of disease, drug type, and administration route and term, and may be appropriately selected by those skilled in the related art. However, in order to accomplish desired effects, the compound of the present invention may be administered in an amount of 0.00001 to 1000 mg/kg per day, preferably, 0.001 to 10 mg/kg per day. The compound may be administered once or separately by several times per day. The dosing amount is not particularly limited within the scope of the present invention from every point of consideration.

In the pharmaceutical composition for dermal and topical use, a mixing amount of the compound according to the present invention may range from 0.0001 to 20 wt. %, preferably, 0.001 to 10 wt. % relative to a total weight of the composition. If the amount is less than 0.001 wt. %, it may be concerned about reduction in effects. When the amount exceeds 20 wt. %, an increase in effects is almost not expected even when the compound is mixed.

With regard to a pharmaceutical administration manner, the compound of the present invention may be used as a pharmaceutically acceptable salt thereof, and can be used alone or in combination with and/or simple gathering with any other pharmaceutically active compound.

The present invention provides a composition for prevention of hair loss and promotion of hair growth, including the compound represented by Formula 1 or a pharmacologically acceptable salt as an effective ingredient.

The composition including the compound represented by Formula 1 or a pharmacologically acceptable salt as an effective ingredient may be used in a variety of applications including, for example, cosmetics for improving dermal inflammation and face cleanser, etc. Products to which the inventive composition can be added may include, for example, cosmetics such as cream, lotion, skin, etc.; cleansing agents; face cleansers; soaps; treatments; solutions for beauty, or the like.

The cosmetics of the present invention may contain a composition selected from a group consisting of water-soluble vitamins, oil-soluble vitamins, polymer peptides, polysaccharides, sphingolipids and seaweeds extracts.

The water-soluble vitamins are not particularly limited but may include any vitamin so far as it can be mixed with cosmetics. Preferably, vitamin B1, vitamin B2, vitamin B6, pyridoxine, pyridoxine hydrochloride, vitamin B12, pantothenic acid, nicotinic acid, nicotinic acid amide, folic acid, vitamin C, vitamin H, or the like may be used, and their salts (thiamine hydrochloride, sodium ascorbate, etc.) or derivatives (ascorbic acid-2-sodium phosphate, ascorbic acid-2-magnesium phosphate, etc.) may also be included in the water-soluble vitamins useable in the present invention. The water-soluble vitamin may be prepared by any conventional method such as microbial transformation, purification of a microbial culture medium, use of enzyme, chemical synthesis, or the like.

The oil-soluble vitamins are not particularly limited but may include any vitamin so far as it can be mixed with cosmetics. Preferably, vitamin A, carotene, vitamin D2, vitamin D3, vitamin E (d1-α-tocopherol, d-α-tocopherol), or the like may be used, and their derivatives (ascorbyl palmitate, ascorbyl stearate, ascorbyl dipalmitate, d1-α-tocopherol acetate, d1-α-tocopherol nicotinate, vitamin E, DL-pantothenyl alcohol, D-pantothenyl alcohol, pantothenyl ethylether, etc.) may also be included in the oil-soluble vitamins useable in the present invention. The oil-soluble vitamin may be prepared by any conventional method such as microbial transformation, purification of a microbial culture medium, use of enzyme, chemical synthesis, or the like.

The polymer peptides are not particularly limited but may include any one so far as it can be mixed with cosmetics. Preferably, collagen, hydrolyzed collagen, gelatin, elastin, hydrolyzed elastin, keratin, or the like may be included. The polymer peptide may be purified and obtained by any conventional method such as purification of a microbial culture medium, use of enzyme, chemical synthesis, or the like. Alternatively, it may be generally obtained by purification of natural substances such as inner skin of a cow or pig, fibroin of silkworm, or the like.

The polysaccharides used herein are not particularly limited but may include any one so far as it can be mixed with cosmetics. Preferably, hydroxyethyl cellulose, xanthan gum, sodium hyaluronate, chondroitin sulfate or salts thereof (sodium salt, etc.), or the like may be included. For example, chondroitin sulfate or its salt used herein may generally be purified from mammals or fishes.

Sphingolipids are not particularly limited but may include any one so far as it can be mixed with cosmetics. Preferably, ceramide, phytosphingosine, sphingoglycolipid, or the like may be included. The sphingolipid used herein may be generally purified from mammals, fishes, shellfishes, yeast or vegetables by any conventional method or prepared by chemical synthesis.

The seaweeds extracts are not particularly limited but may include any one so far as it can be mixed with cosmetics. Preferably, brown algae extract, red algae extract, green algae extract, or the like may be included. Further, carrageenan, alginic acid, sodium alginate, potassium alginate, etc. may also be included in the seaweeds extracts used in the present invention. The seaweed extract may be obtained by purification according to any conventional method.

The cosmetics prepared according to the present invention may optionally include additional components mixed in conventional cosmetic products in addition to the above essential ingredients, if desired.

The components possibly mixed and added to the essential ingredients may include, for example, lipid components, humectants, emollient agents, surfactants, organic or inorganic pigments, organic powder, UV absorbents, preservatives, sanitizers, antioxidants, vegetable extract, pH adjuster, alcohol, colorants, flavors, blood circulation promoter, cooling agents, adiaphoretics, purified water, or the like.

The lipid components may include, for example, ester lipids, hydrocarbon lipids, silicon lipids, fluorine lipids, animal fat, vegetable oil, or the like.

The ester lipids may include, for example, glyceryl tri 2-ethylhexanoate, cetyl 2-ethylhexanoate, isopropyl myristate, butyl myristate, isopropyl palmitate, ethyl stearate, octyl palmitate, isocetyl isostearate, butyl stearate, ethyl linolate, isopropyl linolate, ethyl oleate, isocetyl myristate, isostearyl myristate, isostearyl palmitate, octyldodecyl myristate, isocetyl isostearate, diethyl sebacate, diisopropyl adipate, isoalkyl neopentanate, tri(capryl, capric acid)glyceryl, trimethylolpropane tri 2-ethylhexanoate, trimethylolpropane triisostearate, pentaerythritol tetra 2-ethylhexanoate, cetyl caprylate, decyl laurate, hexyl laurate, decyl myristate, myristyl myristate, cetyl myristate, stearyl stearate, decyl oleate, cetyl lisinooleate, isostearyl laurate, isotridecyl myristate, isocetyl palmitate, octyl stearate, isocetyl stearate, isodecyl oleate, octyldodecyl oleate, octyldodecyl linolate, isopropyl isostearate, cetostearyl 2-ethylhexanoate, stearyl 2-ethylhexanoate, hexyl isostearate, ethyleneglycol dioctanoate, ethyleneglycol dioleate, propyleneglycol dicaprinate, propyleneglycol di(caprylate, caprinate), propyleneglycol dicaprylate, neopentylglycol dicaprinate, neopentylglycol dioctanoate, glyceryl tricaprylate, glyceryl triundecylate, glyceryl triisopalmitate, glyceryl triisostearate, octyldodecyl neopentanoate, isostearyl octanoate, octyl isononanoate, hexyldecyl neodecanoate, octyldodecyl neodecanoate, isocetyl isostearate, isostearyl isostearate, octyldecyl isostearate, polyglycerin ester oleate, polyglycerin ester isostearate, triisocetyl citrate, triisoalkyl citrate, triisooctyl citrate, lauryl lactate, myristyl lactate, cetyl lactate, octyldecyl lactate, triethyl citrate, acetyltriethyl citrate, acetyltributyl citrate, trioctyl citrate, diisostearyl malate, 2-ethylhexyl hydroxystearate, di 2-ethylhexyl succinate, diisobutyl adipate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stealoyl hydroxystearate, stearyl 12-stealoyl hydroxystearate, isostearyl 12-stealoyl hydroxystearate, or the like.

The hydrocarbon lipids may include, for example, squalene, liquid paraffin, alpha-olefin oligomer, isoparaffin, serecine, paraffin, liquid isoparaffin, polybutene, microcrystalline wax, Vaseline, or the like.

The silicon lipids may include, for example, polymethyl silicon, methylphenyl silicon, methyl cyclopolysiloxane, octamethyl polysiloxane, decamethyl polysiloxane, dodecamethyl cyclosiloxane, dimethylsiloxane/methylcetyloxysiloxane copolymer, dimethylsiloxane/methylstealoxysiloxane copolymer, alkyl-modified silicon oil, amino-modified silicon oil, or the like.

The fluorine lipids may include, for example, perfluoropolyether, or the like.

Animal fat or vegetable oil may include, for example, avocado oil, almond oil, olive oil, sesame oil, rice bran oil, safflower oil, soybean oil, corn oil, rape flower oil, apricot kernel oil, palm nucleus oil, palm oil, castor oil, sunflower oil, grape seed oil, cotton seed oil, coconut oil, tallow nut oil, wheat germ oil, rice germ oil, Shea butter, evening primrose oil, macadamia nut oil, meadow foam seed oil, yolk oil, beef tallow, hemp seed oil, mink oil, orange roughy oil, jojoba oil, canderila wax, carnauba wax, liquid lanolin, dehydrated castor oil, or the like.

The humectants may include, for example, water-soluble low molecular humectants, oil-soluble molecular humectants, water-soluble polymer, oil-soluble polymer, or the like.

The water-soluble low molecular humectants may include, for example, serine, glutamine, sorbitol, mannitol, pyrrolidone-sodium carboxylate, glycerin, propyleneglycol, 1,3-butyleneglycol, ethyleneglycol polyethyleneglycol B (degree of polymerization; n=2 or higher), polypropyleneglycol (degree of polymerization; n=2 or higher), polyglycerin B (degree of polymerization; n=2 or higher), lactic acid, lactates, or the like.

The oil-soluble low molecular humectants may include, for example, cholesterol, cholesterol ester, or the like.

The water-soluble polymer may include, for example, carboxyvinyl polymer, polyasparaginic acid salts, tragacanth, xanthan gum, methyl cellulose, hydroxymethyl cellulose, hydroxylethyl cellulose, hydroxypropyl cellulose, carboxymethyl cellulose, water-soluble chitin, chitosan, dextrin, or the like.

The oil-soluble polymer may include, for example, polyvinyl pyrrolidone/eicosen copolymer, polyvinyl pyrrolidone/hexadecen copolymer, nitrocellulose, dextrin fatty acid ester, silicone polymer, or the like.

The emollient agents may include, for example, long chain cholesterylester acylglutamate, cholesteryl hydroxystearate, 12-hydroxystearic acid, stearic acid, rosin acid, lanolin fatty acid cholesteryl ester, or the like.

The surfactants may include, for example, non-ionic surfactants, anionic surfactants, cationic surfactants, amphoteric surfactants, or the like.

The non-ionic surfactants may include, for example, self-emulsion type glycerin monostearate, propyleneglycol fatty acid ester, glycerin fatty acid ester, polyglycerin fatty acid ester, sorbitan fatty acid ester, polyoxyethylene(POE) sorbitan fatty acid ester, POE sorbit fatty acid ester, POE glycerin fatty acid ester, POE alkylether, POE fatty acid ester, POE dehydrated castor oil, POE castor oil, polyoxyethylene/polyoxypropylene (POE/POP) copolymer, POE/POP alkylether, polyether-modified silicone, alkanolamide laurate, alkylamine oxide, hydrated soy phospholipid, or the like.

The anionic surfactants may include, for example, fatty acid soap, α-acylsulfonate, alkylsulfonate, alkylallyl sulfonate, alkylnaphthalene sulfonate, alkyl sulfate, POE alkylether sulfate, alkylamide sulfate, alkyl phosphate, POE alkyl phosphate, alkylamide phosphate, alkyloyl alkyltaurin salts, N-acylamino acid salts, POE alkylether carboxylate, alkyl sulfosuccinate, sodium alkyl sulfoacetate, acylated hydrolyzed collagen peptide salts, perfluoroalkyl ester phosphate, or the like.

The cationic surfactants may include, for example, alkyltrimethylammonium chloride, stearyltrimethylammonium chloride, steraryltrimethylammonium bromide, cetostearyl trimethylammonium chloride, distearyl dimethylammonium chloride, stearylaryl dimethylbenzylammonium chloride, behenyltrimethylammonium bromide, benzalkonium chloride, diethylaminoethylamide stearate, dimethylaminopropylamide stearate, quaternary ammonium salt of lanolin derivative, or the like.

The amphoteric surfactants may include, for example, carboxybetaine, amidebetaine, sulfobetaine, hydroxysulfobetaine, amidesulfobetaine, phosphobetaine, aminocarboxylate, imidazoline derivatives and/or amideamine-based amphoteric surfactants.

The organic and inorganic pigments may include, for example: inorganic pigments such as silicic acid, anhydrous silicic acid, magnesium silicate, talc, sericite, mica, kaolin, bengalra, clay, bentonite, titanium dioxide-coated mica, bismuth oxychloride, zirconium oxide, magnesium oxide, zinc oxide, titanium oxide, aluminum oxide, calcium sulfate, barium sulfate, magnesium sulfate, calcium carbonate, magnesium carbonate, iron oxide, ultramarine, chromium oxide, chromium hydroxide, calamine, and any combination thereof; organic pigments such as polyamide, polyester, polypropylene, polystyrene, polyurethane, vinyl resin, urea resin, phenol resin, fluorine resin, silicon resin, acryl resin, melamine resin, epoxy resin, polycarbonate resin, divinyl benzene/styrene copolymer, silk powder, cellulose, CI pigment yellow, CI pigment orange, etc.; and composite pigments of inorganic and organic pigments, or the like.

The organic powder may include, for example: metallic soap such as calcium stearate; metal salts of alkyl phosphoric acid such as zinc sodium cetylate, zinc laurylate, calcium laurylate, etc.; polymetallic salts of acylamino acid such as calcium N-lauroyl-beta-alanine, zinc N-lauroyl-beta-alanine, calcium N-lauroylglycine, etc.; polymetallic salts of amide sulfonate such as calcium N-lauroyl-taurine, calcium N-palmitoyl-taurine, etc.; N-acyl alkaline amino acid such as N-epsilon-lauroyl-L-lysine, N-epsilon-palmitoyl lysine, N-α-palmitoylol nitin, N-α-lauroyl arginine, N-α-dehydrated tallow fatty acid acyl arginine, etc.; N-acyl polypeptides such as N-lauroyl glycylglycine, etc.; α-amino fatty acid such as α-aminocaprylic acid, α-aminolauric acid, etc.; polyethylene, polypropylene, nylon, polymethylmethacrylate, polystyrene, divinylbenzene/styrene copolymer, ethylene tetrafluoride, or the like.

The UV absorbents may include, for example, para-aminobenzoic acid, ethyl para-aminobenzoate, amyl para-aminobenzoate, octyl para-aminobenzoate, ethyleneglycol salicylate, phenyl salicylate, octyl salcylate, benzyl salicylate, butylphenyl salicylate, homomentyl salicylate, benzyl cinnamate, para-methoxycinnamic acid-2-ethoxylethyl, octyl paramethoxycinnamate, mono-2-ethylhexaneglyceryl diparamethoxycinnamate, isopropyl paramethoxycinnamate, diisopropyl/diisopropyl cinnamic acid ester mixture, urocanic acid, ethyl urocanate, hydroxymethoxybenzophenone, hydroxymethoxybenzophenone sulfonic acid and salts thereof, dihydroxymethoxybenzophenone, sodium dihydroxymethoxybenzophenone disulfonate, dihydroxybenzophenone, tetrahydroxybenzophenone, 4-tert-butyl-4′-methoxydibenzoylmethane, 2,4,6-trianilino-p-(carbo-2′-ethylhexyl-1′-oxy)-1,3,5-triazine, 2-(2-hydroxy-5-methylphenyl)benzotriazole, or the like.

The sanitizers may include, for example, hinokitiol, trichloric acid, trichlorohydroxydiphenylether, chlorohexidine gluconate, phenoxyethanol, resorcine, isopropylmethylphenol, azulene, salicylic acid, zinc pyrithione, benzalkonium chloride, light sensitive element No. 301, sodium mononitroguaiacol, undecylenic acid, or the like.

The antioxidants may include, for example, butylhydroxyanisole, propyl gallate, elisorbic acid, or the like.

The pH adjuster may include, for example, citric acid, sodium citrate, malic acid, sodium malate, fumaric acid, sodium fumarate, succinic acid, sodium succinate, sodium hydroxide, sodium monohydrophosphate, or the like.

The alcohol used herein may include, for example, higher alcohols such as cetyl alcohol.

Further, additional components possibly mixed with the inventive composition are not particularly limited to the above substances, and any one among the above substances can be mixed within a range in which it does not affect the purposes and effects of the present invention. This component may be mixed with the composition in a mixing amount of, preferably, 0.01 to 5 wt. % and, more preferably, 0.01 to 3 wt. % to a total weight of the composition.

The cosmetic formulation of the present invention may have a form of solution, emulsion, viscous mixture, etc.

The cosmetic composition of the present invention may contain components generally used in any conventional cosmetic composition, in addition to the effective ingredient, that is, the compound represented by Formula 1. For example, typical adjuvants and carriers such as stabilizers, solvates, vitamins, pigments and flavors may be included.

The cosmetic composition of the present invention can be prepared in any of formulations generally produced in the related art, for example, emulsion, cream, beauty wash, pack, foundation, lotion, solution for beauty, hair cosmetics, or the like.

More particularly, the cosmetic composition of the present invention may be prepared in any of formulations including, for example, skin lotion, skin softener, skin toner, astringent, lotion, milk lotion, moisturizing lotion, nourishing lotion, massage cream, nourishing cream, moisturizing cream, hand cream, foundation, essence, nourishing essence, pack, soap, cleansing foam, cleansing lotion, cleansing cream, body lotion and body cleanser.

If the formulation of the present invention is any of paste, cream or gel, animal fibers, vegetable fibers, wax, paraffin, starch, tragacanth, cellulose derivatives, polyethyleneglycol, silicone, bentonite, silica, talc or zinc oxide may be used as a carrier component.

If the inventive formulation is a powder or spray, lactose, talc, silica, aluminum hydroxide, calcium silicate or polyamide powder may be used as a carrier component. More particularly, in the case of a spray type formulation, it may further include a propellant such as chlorofluorohydrocarbon, propane/butane or dimethylether.

Alternatively, if the inventive formulation is a solution or emulsion, a solvent, solvate or emulsifier may be used as a carrier component. For example, it may include water, ethanol, isopropanol, ethyl carbonate, ethyl acetate, benzyl alcohol, benzyl benzoate, propyleneglycol, 1,3-butylglycol oil, glycerol aliphatic ester, polyethyleneglycol or fatty acid ester of sorbitan.

If the inventive formulation is a suspension, a carrier component including, for example: a liquid diluent such as water, ethanol or propyleneglycol; a suspension such as ethoxylated isostearyl alcohol, polyoxyethylene sorbitol ester and polyoxyethylene sorbitan ester; microcrystalline cellulose; aluminum metahydroxide; bentonite; agar or tragacanth, or the like, may be used.

If the inventive formulation is a surfactant-containing cleansing agent, aliphatic alcohol sulfate, aliphatic alcohol ether sulfate, monoester sulfosuccinate, isethionate, imidazolinium derivatives, methyltaurate, sarcosinate, fatty acid amide ether sulfate, alkyl aminobetaine, aliphatic alcohol, fatty acid glyceride, fatty acid diethanolamide, vegetable oil, linoline derivatives or ethoxylated glycerol fatty acid ester may be used as a carrier component.

The compound represented by Formula 1 contained as an effective ingredient with activity of preventing hair loss and promoting hair growth in the composition according to the present invention may be contained in a content of 0.00001 to 10 wt. % to a total weight of the composition.

The composition of the present invention may include the compound represented by Formula 1 as an effective ingredient, as well as additional components generally used in the related art including, for example, common adjuvants and carriers such as antioxidants, stabilizers, solvates, vitamins, pigments and/or flavors.

The composition of the present invention may be prepared in any of formulations generally produced in the related art including, for example, solution, suspension, emulsion, paste, gel, cream, lotion, powder, soap, surfactant-containing cleansing solution, oil, powdery foundation, emulsion type foundation, wax foundation and spray, however, it is not limited thereto. Further, when the composition of the present invention is commonly available in a cosmetic composition, it can be prepared in any of formulations including, for example, emollient beauty wash, nourishing beauty wash, nourishing cream, massage cream, essence, eye cream, cleansing cream, cleansing foam, cleansing water, pack, spray or powder.

If the inventive formulation is a paste, cream or gel, animal oil, vegetable oil, wax, paraffin, starch, tragacanth, cellulose derivatives, polyethyleneglycol, silicone, bentonite, silica, talk or zinc oxide may be used as a carrier component.

If the inventive formulation is a powder or spray, lactose, talc, silica, aluminum hydroxide, calcium silicate or polyamide powder may be used as a carrier component. More particularly, in case of a spray type formulation, the formulation may further include a propellant such as chlorofluorohydrocarbon, propane/butane or dimethylether.

If the inventive formulation is a solution or emulsion, a solvent, solvate or emulsifier may be used as a carrier component. For example, the formulation may include, for example, water, ethanol, isopropanol, ethyl carbonate, ethyl acetate, benzyl alcohol, benzyl benzoate, propyleneglycol, 1,3-butyleneglycol, glycerol aliphatic ester, polyethyleneglycol or fatty acid ester of sorbitan.

If the inventive formulation is a suspension, aliphatic alcohol sulfate, aliphatic alcohol ether sulfate, monoester sulfosuccinate, isethionate, imidazolinium derivatives, methyltaurate, sarcosinate, fatty acid amide ether sulfate, alkyl amidobetaine, aliphatic alcohol, fatty acid glyceride, fatty acid diethanolamide, vegetable oil, lanolin derivatives, ethoxylated glycerol fatty acid ester, or the like may be used as a carrier component.

1. A composition for prevention of hair loss and promotion of hair growth, comprising: at least one selected from compounds represented by Formula 1 below as an effective ingredient: 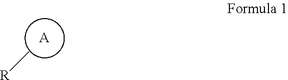
wherein 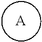
is derived from polycyclic compounds, and R is a hydroxyl group, or saturated or unsaturated straight or branched alkyloxy or acyloxy group having 1 to 20 carbon atoms.

2. The composition according to claim 1, wherein the compound represented by Formula 1 is represented by Formula 2 below, 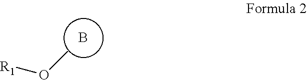
wherein 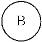
has a mother nucleus structure of at least four (4) rings linked together, and R1 is hydrogen, or a saturated or unsaturated C₁₋₃₀ straight or branched alkyl or acyl group.

3. The composition according to claim 1, wherein the compound represented by Formula 1 is at least one selected from a group consisting of β-sitosteryl stearate, desmosteryl stearate, stigmasteryl stearate, campesteryl stearate, brassicasteryl stearate, fucosteryl stearate, cholesteryl stearate, stearoyl dehydroandrosterone, stearoyl α-amyrin, stearoyl β-amyrin, stearoyl α-boswellic acid, stearoyl β-boswellic acid, stearoyl ursolic acid, stearoyl oleanolic acid, stearoyl glycyrrhetinic acid and stearoyl betulinic acid.

4. The composition according to claim 1, wherein the compound represented by Formula 1 is contained in an amount of 0.0001 to 10 wt. % to a total weight of the composition.
